(19) United States
(12) Patent Application Publication
(10) Pub. No.: US 2026/0188469 A1
(43) Pub. Date: Jul. 2, 2026

(54) MEDICAL IMAGE DIAGNOSTIC DEVICE, MEDICAL INFORMATION PROCESSING DEVICE, MEDICAL INFORMATION PROCESSING SYSTEM, MEDICAL INFORMATION PROCESSING METHOD, AND PROGRAM

(71) Applicant: Canon Kabushiki Kaisha, Tokyo (JP)

(72) Inventors: Daigo OKUHATA, Sakura (JP); Hiroyuki YAMASAKI, Yaita (JP); Ryoichi ONO, Utsunomiya (JP); Taro TAKOSHIMA, Nasushiobara (JP)

(73) Assignee: Canon Kabushiki Kaisha, Tokyo (JP)

(21) Appl. No.: 19/431,099

(22) Filed: Dec. 23, 2025

(30) Foreign Application Priority Data
Dec. 27, 2024 (JP) 2024-232368

(51) Int. Cl. G16H 30/40; G16H 10/60; G16H 50/20

(52) U.S. Cl. CPC G16H 30/40; G16H 10/60; G16H 50/20

(57) ABSTRACT

A medical information processing device of embodiments includes processing circuitry. The processing circuitry acquires update trigger information related to an update of a workflow for processing medical information, and updates the workflow on the basis of the update trigger information.

### CROSS-REFERENCE TO RELATED APPLICATION

The present application claims priority based on Japanese Patent Application No. 2024-232368 filed December 27, 2024, the content of which is incorporated herein by reference.

### FIELD

The embodiments disclosed in this specification and drawings relate to a medical image diagnostic device, a medical information processing device, a medical information processing system, a medical information processing method, and a program.

### BACKGROUND

Medical information acquired by a modality, such as medical images captured by a medical image diagnostic device, is transmitted to a medical information processing device. The medical information processing device performs processing such as analyzing the received medical images according to a workflow, and the like. A workflow for processing a medical image is determined, for example, by an identification label attached to the medical image, or the like. A workflow may include, for example, one or more analysis applications (hereinafter referred to as apps). Workflow settings define a processing flow of an analysis app and the relationship between input and output data.

Analysis apps are developed daily, with new analysis apps being released and existing analysis apps being upgraded. Incorporating such new analysis apps into a workflow can improve workflow efficiency, but it can be difficult to determine how to incorporate a new analysis application into a workflow to achieve significant effects, making it difficult to improve the efficiency of analysis work.

### BRIEF DESCRIPTION OF THE DRAWINGS

FIG. 1 is a block diagram showing an example of a configuration of a medical information processing system 1.

FIG. 2 is a diagram for describing a medical image analysis flow in the medical information processing system 1.

FIG. 3 is a diagram showing an example of a configuration of a medical information processing device 100.

FIG. 4 is a diagram showing an example of the contents of a workflow DB 151.

FIG. 5 is a flowchart showing an example of processing of the medical information processing device 100.

FIG. 6 is a flowchart showing an example of processing of the medical information processing device 100.

FIG. 7 is a flowchart showing an example of processing of the medical information processing device 100.

FIG. 8 is a diagram showing an example of display contents of a display 130.

FIG. 9 is a flowchart showing another example of the processing of the medical information processing device 100.

### DETAILED DESCRIPTION

Hereinafter, a medical image diagnostic device, a medical information processing device, a medical information processing system, a medical information processing method, and a program of embodiments will be described with reference to the drawings.

The medical information processing device of embodiments includes processing circuitry. The processing circuitry acquires update trigger information related to update of a workflow for processing medical information, and updates the workflow on the basis of the update trigger information.

### (First Embodiment)

FIG. 1 is a block diagram showing an example a configuration of a medical information processing system 1. The medical information processing system 1 includes, for example, a hospital information system (HIS) 10, a radiology information system (RIS) 20, a medical image diagnostic device (modality) 30, a picture archiving and communication system (PACS) 40, and a medical information processing device 100. The medical information processing system 1 is also referred to as an in-hospital system.

The HIS 10, the RIS 20, the modality 30, the PACS 40, and the medical information processing device 100 in the medical information processing system 1 can communicate with each other via a network NW such as a local area network (LAN). The network NW may include the Internet, a cellular network, a Wi-Fi network, a wide area network (WAN), etc., instead of or in addition to a LAN. The RIS 20, the modality 30, and the PACS 40 are examples of external devices.

The HIS 10 is a computer system that supports operations within a hospital. Specifically, the HIS 10 has various subsystems. These subsystems include, for example, an electronic medical record system, a medical accounting system, a medical appointment system, a hospital reception system, and an admission and discharge management system.

The HIS 10 is a computer system that supports operations within a hospital. Specifically, the HIS 10 has various subsystems. The various subsystems include, for example, an electronic medical record system, a medical accounting system, a medical appointment system, a hospital reception system, and an admission and discharge management system.

The HIS 10 includes computers such as server devices and client terminals equipped with a processor such as a central processing unit (CPU), memories such as a read only memory (ROM) and a random access memory (RAM), a display, an input interface, and a communication interface.

Users use the electronic medical record system included in the HIS 10 to input and view patient information. Users issue imaging examination orders to the HIS 10. The HIS 10 transfers order information corresponding to imaging examination orders to other systems such as the RIS 20.

The RIS 20 is a computer system that supports operations in an imaging diagnostic department. The RIS 20 performs linking reservation information to examination equipment, management of examination information, and the like in addition to management of reservations for imaging examination orders in cooperation with the HIS 10. The RIS 20 includes, for example, computers such as server devices and client terminals equipped with a processor such as a CPU, memories such as a ROM and a RAM, a display, an input interface, and a communication interface.

The RIS 20 is a management device that creates examination workflows that indicate procedures for examining patients (subjects) within a hospital and manages the created examination workflows. The RIS 20 updates examination workflows having processing procedures that change due to, for example, new workflows provided by the medical information processing device 100. An examination workflow is an example of an external workflow.

The modality 30 performs imaging (photographing) according to imaging conditions (imaging protocols) determined on the basis of imaging examination instructions and the like, for example. Examples of the modality 30 include X-ray computed tomography (X-ray CT) devices, X-ray diagnostic devices, magnetic resonance imaging devices, ultrasound diagnostic devices, and nuclear medicine diagnostic devices. Medical images include, for example, X-ray CT images, magnetic resonance images, and echo images.

The modality 30 is operated, for example, by a user. Medical images (image data) generated by imaging performed by the modality 30 are transmitted to the PACS 40 or the medical information processing device 100. The modality 30 executes an examination to acquire examination information related to the examination. The examination information is, for example, a medical image, and execution of the examination is, for example, capturing of a medical image. The modality 30 is an example of an examination device.

The PACS 40 is a computer system that receives medical images transmitted by the modality 30 and the medical information processing device 100 and stores the same in a database. In response to requests from clients, the PACS 40 transmits (transfers) medical images stored in the database.

The PACS 40 includes a server computer that includes a processor such as a CPU, memories such as a ROM and a RAM, a display, an input interface, and a communication interface. The PACS 40 stores a plurality of medical images. The plurality of medical images includes, for example, medical images of a plurality of subjects captured at different times. The PACS 40 is an example of an image storage device.

The configuration of the in-hospital system H is not limited to the above. The in-hospital system H may also include, for example, a radiology report creation device. Furthermore, several elements of the in-hospital system H may be integrated. For example, the HIS 10 and the RIS 20 may be integrated into a single system.

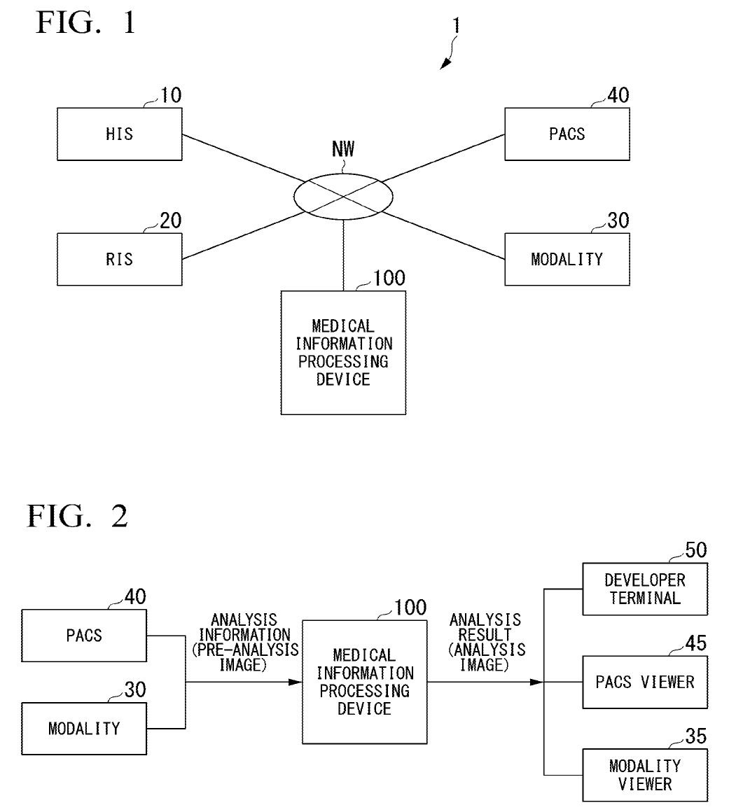

The medical information processing system 1 is a system that performs image processing on medical images captured by the modality 30 to generate analysis results (analysis images) and provide the analysis results to the modality 30 or the PACS 40. FIG. 2 is a diagram for describing a flow of medical image analysis in the medical information processing system 1.

In the medical information processing system 1, first, a medical image (pre-analysis image) captured by the modality 30 or stored in the PACS 40 is transmitted as analysis information from the modality 30 or the PACS 40 to the medical information processing device 100. The medical information processing device 100 analyzes the transmitted pre-analysis image using image processing to generate an analysis image as an analysis result. The medical information processing device 100 transmits the generated analysis image to a developer terminal 50 used by a developer, the PACS 40, the modality 30, etc.

A viewer included in the developer terminal 50, a PACS viewer 45 included in the PACS 40, and a modality viewer 35 included in the modality 30 each display the transmitted analysis images on a monitor or a display. The developer using the developer terminal 50, and hospital staff, technicians, doctors, and others using the PACS 40 and the modality 30 can view the analysis image displayed on the display or the monitor using the PACS viewer 45 or the modality viewer 35.

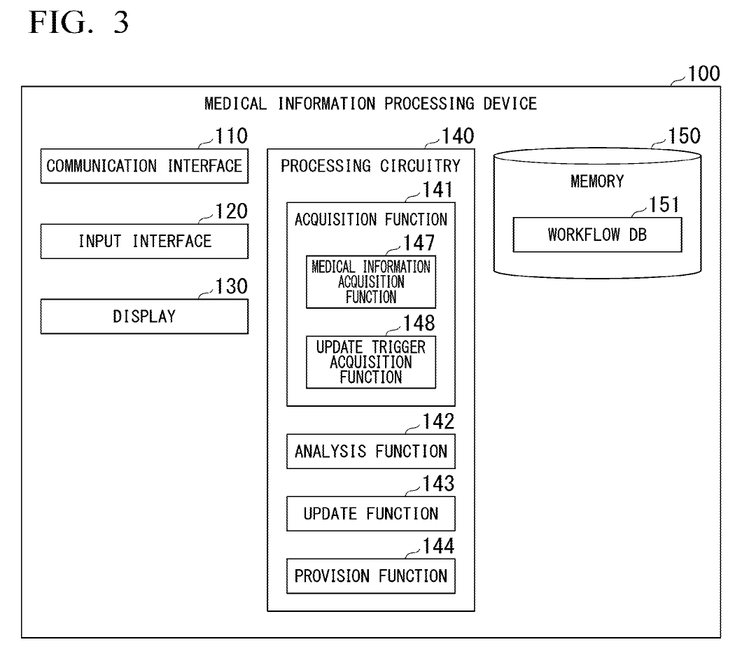

Subsequently, the medical information processing device 100 will be described. FIG. 3 is a diagram showing an example of a configuration of the medical information processing device 100. The medical information processing device 100 includes, for example, a communication interface 110, an input interface 120, a display 130, processing circuitry 140, and a memory 150.

The communication interface 110 communicates with an external device such as a client terminal 200 on a LAN or the like via a network NW (FIG. 1). The communication interface 110 includes, for example, a communication interface such as a network interface card (NIC).

The input interface 120 receives various input operations from an administrator or the like that manages the medical information processing device 100. The input interface 120 converts the received input operations into electrical signals and outputs the electrical signals to the processing circuitry 140. In a case where an input operation is performed by the administrator or the like, the input interface 120 generates information corresponding to the input operation. The input interface 120 outputs the generated information corresponding to the input operation to the processing circuitry 140.

The input interface 120 includes, for example, a mouse, a keyboard, a trackball, a switch, a button, a joystick, a touch panel, and the like. The input interface 120 may also be a user interface that receives audio input from a microphone and the like. In a case where the input interface 120 is a touch panel, the input interface 120 may also have the display function of the display 130.

Note that in this specification, the input interface is not limited to interfaces equipped with physical operating components such as a mouse and a keyboard. For example, examples of the input interface also include electrical signal processing circuitry that receives an electrical signal corresponding to an input operation from an external input device provided separately from the device and outputs this electrical signal to the processing circuitry 140.

The display 130 displays various types of information. For example, the display 130 displays images generated by the processing circuitry 140 and a graphical user interface (GUI)) for receiving various input operations from an operator. For example, the display 130 is a liquid crystal display (LCD), a cathode ray tube (CRT) display, or an organic electroluminescence (EL) display.

The processing circuitry 140 includes, for example, an acquisition function 141, an analysis function 142, an update function 143, and a provision function 144. The processing circuitry 140 realizes these functions by, for example, a hardware processor (computer) executing programs stored in the memory (storage circuitry) 150.

The hardware processor refers to circuitry such as a CPU, a graphics processing unit (GPU), an application-specific integrated circuit (ASIC), or a programmable logic device (e.g., a simple programmable logic device (SPLD) or a complex programmable logic device (CPLD), or a field programmable gate array (FPGA)).

A program may be directly embedded in the circuit of the hardware processor instead of being stored in the memory 150. In this case, the hardware processor realizes functions by reading and executing the program embedded in the circuit. The program may be pre-stored in the memory 150, or may be stored in a non-transitory storage medium such as a DVD or CD-ROM and may be installed in the memory 150 from the non-transitory storage medium by inserting the non-transitory storage medium into a drive device (not shown) of the medical information processing device 100.

The hardware processor is not limited to being configured as a single circuit and may be configured as a single hardware processor by combining a plurality of independent circuits to realize each function. Furthermore, a plurality of components may be integrated into a single hardware processor to realize each function. The hardware processor, the memory, etc. of the medical information processing device 100 are provided separately from a hardware processor, a memory, etc. of the HIS 10, but these may also be shared.

The memory 150 stores a workflow database (hereinafter referred to as DB) 151. The workflow DB 151 stores a plurality of workflows at the time of image processing of medical images by the medical information processing device 100. A workflow is set, for example, for each identification label assigned to a medical image. One of the workflows stored in the workflow DB 151 is assigned to image processing of a medical image.

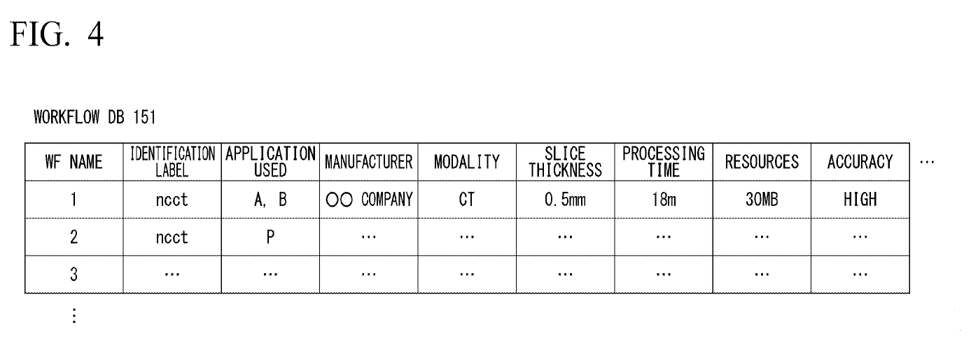

FIG. 4 shows an example of the contents of the workflow DB 151. The workflow DB 151 includes information on each item for each WF name. Examples of items include an identification label, an analysis application used in a workflow (hereinafter referred to as an application used), a manufacturer, a modality, a slice thickness, a processing time, resources, and accuracy. The workflow DB 151 also includes other information such as the cost, the number of executions, and trends, and this information may be updated as appropriate.

The acquisition function 141 includes, for example, a medical information acquisition function 147 and an update trigger acquisition function 148. The medical information acquisition function 147 acquires medical images transmitted by the modality 30 or the PACS 40. The medical information acquisition function 147 acquires supplementary information for supplementing missing data that is lacking when creating a new workflow. The acquisition function 141 is an example of an acquisition unit.

The update trigger acquisition function 148 acquires update trigger information related to update of a medical information processing workflow. The update trigger information includes, for example, change information. The change information includes, for example, at least one piece of information related to change from among the identification label included in the workflow, application used, modality information related to a modality that has acquired the medical information, workflow processing time, resources, accuracy, cost, number of executions, trends, or user reviews. The change information includes change of an examination workflow managed by the RIS 20. The update trigger acquisition function 148 acquires update trigger information, for example, every time a certain period or a designated period elapses.

The analysis function 142 analyzes medical information in accordance with a workflow. The analysis function 142 selects an appropriate workflow for analysis from the workflows included in the workflow DB 151, for example, on the basis of medical images acquired by the medical information acquisition function 147. The analysis function 142 analyzes the medical images using the selected workflow. The analysis function 142 is an example of an analysis unit.

The update function 143 updates a workflow on the basis of update trigger information. For example, in a case where update trigger information is acquired by the update trigger acquisition function 148, the update function 143 updates the workflow on the basis of the acquired update trigger information. Workflow updates include updates that modify some of existing workflows and updates that create a new workflow. The update function 143 is an example of an update unit.

The provision function 144 provides comparison information representing comparison of statuses before and after a workflow update. For example, in a case where the update function 143 updates a workflow or intends to update a workflow, the provision function 144 provides statuses before and after the workflow update by displaying the same on the display 130, outputting the same as audio, or transmitting the same to an external device such as the RIS 20 or a user terminal used by the user.

The comparison information provided by the provision function 144 includes information obtained by comparing at least some of workflow elements before and after the update. The update function 143, for example, switches the workflow assigned to medical image processing on the basis of a response to the comparison information provided by the provision function 144. The provision function 144 is an example of a provision unit.

The update function 143 can create a new workflow to which missing data has been added. In a case where the provision function 144 can request that the RIS 20, the modality 30, the PACS 40, etc. supplement the missing data, the provision function 144 transmits supplement request information for requesting supplement of the missing data to the RIS 20, the modality 30, the PACS 40, etc. Missing data includes, for example, results of an examination performed by the modality 30 and medical images stored in the PACS 40. The supplement request information includes, for example, a request for provision of examination information obtained as a result of an examination performed by the modality 30 and a request for provision of medical images stored in the PACS 40.

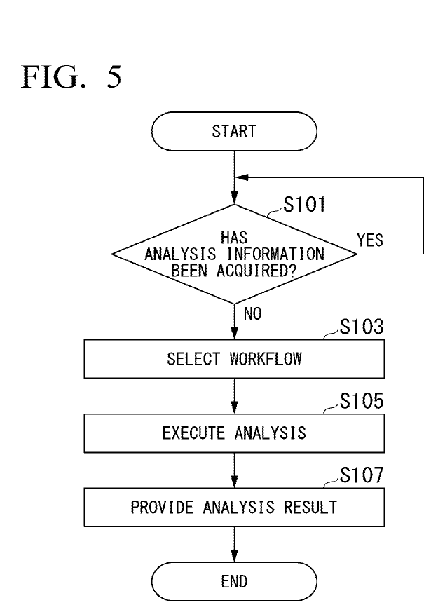

Next, processing of the medical information processing device 100 of an embodiment will be described. FIG. 5 to FIG. 7 are all flowcharts showing an example of processing of the medical information processing device 100. The medical information processing device 100 is a device that analyzes medical images (pre-analysis images) acquired by the modality 30 and stored in the PACS 40. The medical information processing device 100 of the embodiment also has the function of appropriately updating a workflow used to analyze pre-analysis images. Therefore, processing of analyzing medical images in the medical information processing device 100 will first be described with reference to FIG. 5.

In analyzing a medical image, the medical information processing device 100 first determines whether a pre-analysis image which will serve as analysis information to be transmitted by the modality 30 or the PACS 40 has been acquired through the medical information acquisition function 147 (step S101). If it is determined that analysis information has not been acquired, the medical information acquisition function 147 repeats the processing of step S101.

If the medical information acquisition function 147 determines that analysis information has been acquired, the analysis function 142 selects a workflow to be used for analysis on the basis of identification label information assigned to the acquired analysis information (pre-analysis image), or the like (step S103). Selection of a workflow may be performed on the basis of information other than identification label information or any other information that can define a workflow, such as a data type or a digital imaging and communication in medicine (DICOM) tag. Although selection of a workflow is performed by the analysis function 142, a workflow to be selected may be designated by the user operating the input interface 120.

Subsequently, the analysis function 142 performs analysis in accordance with the selected workflow on the pre-analysis image acquired by the medical information acquisition function 147 (step S105) and generates an analysis image. Subsequently, the provision function 144 provides the analysis image generated by the analysis function 142 as an analysis result (step S107). In this manner, the medical information processing device 100 ends the processing shown in FIG. 5.

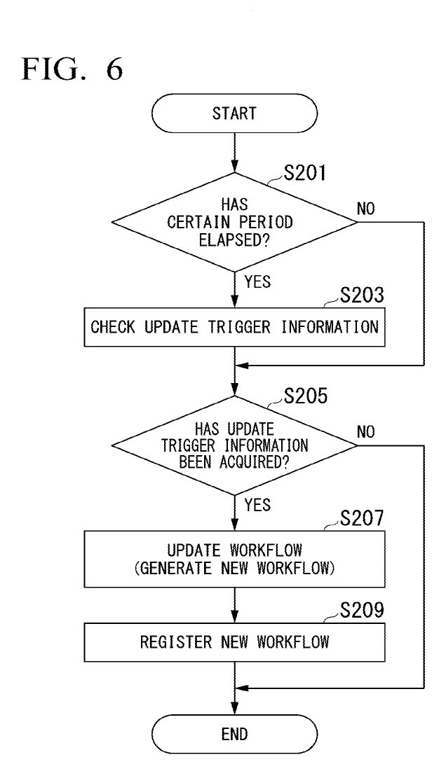

Next, workflow update processing performed by the medical information processing device 100 will be described. FIG. 6 shows a procedure of workflow update processing in the medical information processing device 100. In updating a workflow, the medical information processing device 100 first determines whether a certain period for updating the workflow has elapsed through the update trigger acquisition function 148 (step S201). Any period may be used as the certain period, for example, one day, one week, or one month. Instead of a certain period, a designated period designated by the user may also be used.

If it is determined that the certain period has elapsed, the update trigger acquisition function 148 checks presence or absence of update trigger information (step S203). The update trigger acquisition function 148 checks for update trigger information, for example, by checking with a provider of an alternative analysis application for an application used in the workflow to check update of the application or presence or absence of the analysis application, or by checking for replacement and version upgrade of the modality 30, changes in the workflow or operations within the hospital, etc.

The update trigger acquisition function 148 checks, for example, whether an unpurchased analysis application has been released in an application store in order to check update of the used application or presence or absence of the alternative analysis application. The update trigger acquisition function 148 also checks release of a new version of the application used, a case where common processing such as image alignment is split into a separate analysis application, a case where a post-processing application is integrated with the analysis application, and the like.

Such an analysis application may be checked not only through an application store, but also by any method that can acquire information about the analysis application, such as checking the website of the provider of the analysis application. If it is determined that the certain period has not elapsed, the update trigger acquisition function 148 skips the processing of step S203.

Subsequently, the update trigger acquisition function 148 determines whether update trigger information has been acquired (step S205). In addition to checking the update trigger information in step S203, the update trigger acquisition function 148 also acquires the update trigger information, for example, by receiving notification that the used application has been updated from the provider of the used application.

If the update trigger acquisition function 148 determines that the update trigger information has not been acquired, the medical information processing device 100 ends the processing shown in FIG. 6. If the update trigger acquisition function 148 determines that update trigger information has been acquired, the update function 143 updates the existing workflow and generates a new workflow on the basis of the update trigger information acquired by the update trigger acquisition function 148 (step S207). For example, the update function 143 generates a new workflow such that the performance, resources, and accuracy are improved compared to the existing workflow. The update function 143 may also generate a new workflow by replacing an application used in the existing workflow with an application used that has received favorable reviews from users.

Subsequently, the provision function 144 registers, in the memory 150, the new workflow generated by updating the existing workflow through the update function 143 (step S209). In the memory 150, the workflow DB 151 is updated according to provision of the new workflow, the new workflow is added to the workflow DB 151, and if necessary, the existing workflow before the update is deleted from the workflow DB 151. The medical information processing device 100 then ends the processing shown in FIG. 6.

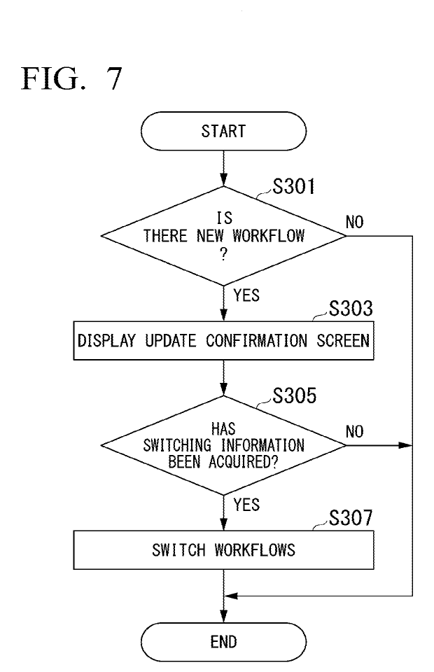

Subsequently, processing in the medical information processing device 100 after a new workflow has been registered will be described with reference to FIG. 7. FIG. 7 shows a processing procedure in the medical information processing device 100 after a new workflow has been registered. The medical information processing device 100 first determines whether a new workflow has been registered through the update function 143 (step S301).

If the update function 143 determines that no new workflow has been registered, the medical information processing device 100 ends the processing shown in FIG. 7. If the update function 143 determines that a new workflow has been registered, the provision function 144 displays an update confirmation screen on the display 130 (step S303). The update confirmation screen is confirmed, for example, by a user operating the medical information processing device 100.

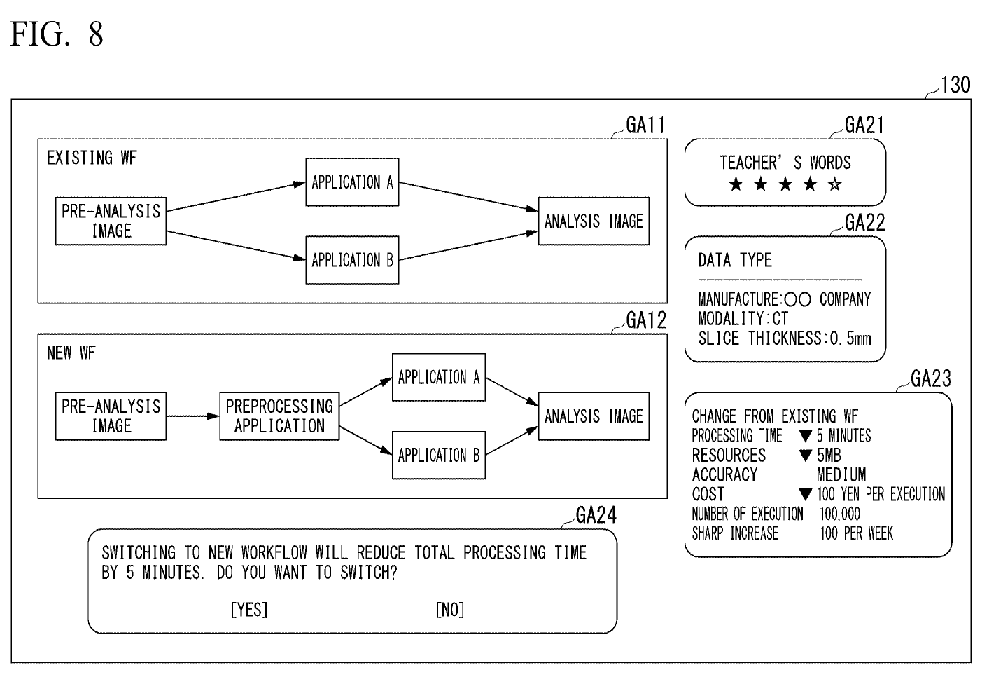

FIG. 8 is a diagram showing an example of display contents on the display 130. The display 130, on which the update confirmation screen is displayed, displays an existing workflow image GA11, a new workflow image GA12, a user review image GA21, a data type image GA22, a change explanation image GA23, and a switching instruction image GA24. The update confirmation screen is comparison information provided by the provision function 144.

The existing workflow image GA11 is an image for explaining a processing flow of the pre-update workflow (existing workflow). The existing workflow image GA11 displays, for example, a flow through which a pre-analysis image is analyzed by applications A and B to generate an analysis image. The new workflow image GA12 is an image for explaining a processing flow of a post-update workflow (new workflow). The new workflow image GA12 displays, for example, a flow through which a pre-analysis image is pre-analysis-processed by a preprocessing application, and then analyzed by the applications A and B to generate an analysis image.

The user review image GA21 displays, for example, ratings and reviews of the new workflow and analysis applications used in the new workflow, such as the applications A and B, by users who have previously used the new workflow and the analysis applications used in the new workflow. In the example shown in FIG. 8, the rating of the new workflow is indicated by the number of stars. The user review image GA21 may also include text data, and the like.

The data type image GA22 displays information such as the name of the company that manufactured the analysis applications and the modality 30 used in the new workflow, the type of the modality 30 used to generate images (capture images), and the slice thickness of captured images. Information related to a data type, and other information may also be displayed in the data type.

The change explanation image GA23 displays, for example, information on the existing workflow, the new workflow, and the amount of change in various items. These items include information of processing time, resources, accuracy, cost, number of executions, and trend. The example shown in FIG. 8 indicates that the processing time has been reduced by 5 minutes, resource usage has been reduced by 5 MB, the accuracy has become medium, and the cost has been reduced by 100 yen per execution. The number of executions displays the number of times the new workflow has been executed (including the number of executions by other hospitals, etc.), and the trend indicates an increase of 100 times per week in the number of uses, displaying a sharp increase in the number of uses.

The switching instruction image GA24 includes information that allows the user to select whether to transmit switching information for switching processing assigned to the existing workflow from the existing workflow to the new workflow to the update function 143. The switching information is information in response to comparison information provided by the provision function 144. The switching instruction image GA24 displays text information such as “Switching to the new workflow will reduce the total processing time by 5 minutes. Do you want to switch?” along with button images for “Yes” and “No.”

The user can designate a button image by operating the input interface 120. The user determines whether to switch the processing assigned to the existing workflow to the new workflow while viewing the update confirmation screen. If the user wishes to switch the processing to the new workflow, the user performs an input operation designating “Yes,” and the input interface 120 transmits switching information to the update function 143 in response to the input operation of the user. If the user wishes to maintain the processing assigned to the existing workflow, the user performs an input operation designating “No.” In this case, switching information is not transmitted, and the existing workflow is maintained.

Returning to the flow shown in FIG. 7, the update function 143 determines whether switching information transmitted by the input interface 120 has been acquired (step S305). If it is determined that switching information has been acquired, the update function 143 switches the workflow assigned to processing of the medical image (pre-analysis image) (step S307). The medical information processing device 100 then ends the processing shown in FIG. 7. If the update function 143 determines that switching information has not been acquired, the medical information processing device 100 simply ends the processing shown in FIG. 7.

The medical information processing device 100 of the first embodiment updates a workflow in a case where update trigger information, such as release of a new analysis application, has been acquired. This makes it possible to generate a new workflow that enables efficient analysis or update an existing workflow. Therefore, the efficiency of analysis work can be improved.

### (Second Embodiment)

Next, a medical information processing device according to a second embodiment will be described. The medical information processing device 100 according to the second embodiment has a configuration similar to that of the medical information processing device 100 according to the first embodiment, with the main difference being processing in the processing circuitry 140. Hereinafter, the medical information processing device 100 according to the second embodiment will be described, focusing on the differences from the first embodiment.

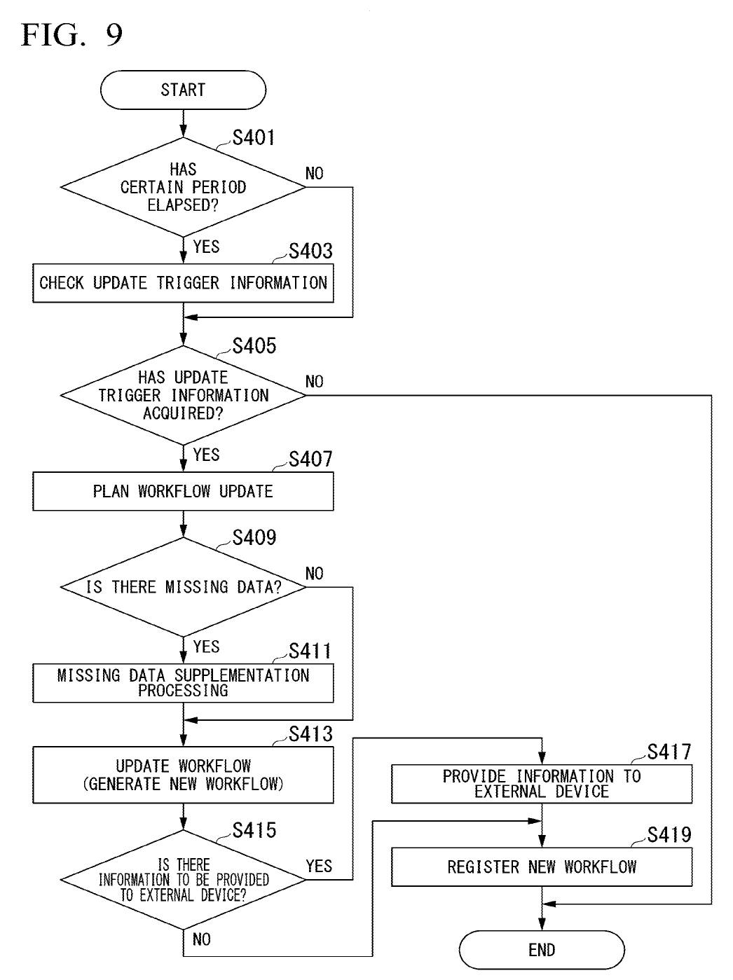

FIG. 9 is a flowchart showing an example of processing performed by the medical information processing device 100 according to the second embodiment. In the medical information processing device 100 according to the second embodiment, the update trigger acquisition function 148 first determines whether a certain period for updating a workflow has elapsed (step S401). If it is determined that the certain period has elapsed, the update trigger acquisition function 148 checks presence or absence of update trigger information (step S403).

If it is determined that the certain period has not elapsed, the update trigger acquisition function 148 skips the processing of step S303. Subsequently, the update trigger acquisition function 148 determines whether update trigger information has been acquired (step S405). If the update trigger acquisition function 148 determines that update trigger information has not been acquired, the medical information processing device 100 ends the processing shown in FIG. 9. Up to this point, the medical information processing device 100 performs the same processing as in the first embodiment.

Subsequently, the update function 143 plans a workflow update (step S407). The workflow update is planned by generating candidate workflows that can become new workflows, for example, using the procedure used to generate a new workflow in the first embodiment. Subsequently, the update function 143 determines whether there is any missing data that will be required to execute a candidate workflow (step S409).

For example, the missing data is related to an example of a case where the application B is executed on the current medical image (result of the current examination) in the existing workflow and the candidate workflow. In this case, it is assumed that the candidate workflow uses examination results of the application A for a past examination that has not been used in the existing workflow. In this case, if the existing workflow does not use the examination result of the application A, the update function 143 determines that the examination result of the application A is missing data. Similarly, if there is data that would enable the candidate workflow to be realized with this data, the update function 143 determines that this data is missing data.

Subsequently, the update function 143 executes processing of supplementing the missing data (step S411). The processing of supplementing the missing data is processing of supplementing processing for acquiring the missing data, and if the examination result of the application A is determined to be missing data, for example, processing of acquiring the past examination results of the application A for a subject being examined from the modality 30 or the PACS 40 is supplemented. The processing of supplementing the missing data may, for example, supplement processing of extracting and acquiring existing past data, or supplement processing of requesting an examination from the modality 30 and acquiring examination results in a case where new data other than existing data needs to be acquired.

If it is determined in step S409 that there is no missing data, the update function 143 skips the processing of step S411. Subsequently, after processing the missing data, the update function 143 updates the workflow by generating the candidate workflow as a new workflow (step S413).

Subsequently, the provision function 144 determines whether there is provision information to be provided to external devices (step S415). The provision information includes, for example, supplement request information for requesting transmission of missing data from the PACS 40 or newly requesting an examination from the modality 30, and a new workflow in a case where an examination workflow of the hospital managed by the RIS 20 is changed due to workflow update. The provision function 144 transmits the supplement request information as provision information to external devices such as the RIS 20, the modality 30, and the PACS 40.

If it is determined that there is provision information, the provision function 144 provides the provision information to the external devices such as the RIS 20, the modality 30, and the PACS by transmitting the same using the communication interface 110 (step S417). If it is determined that there is no provision information, the provision function 144 skips the processing of step S417. The update function 143 then registers the new workflow in the workflow DB 151 (step S419). The medical information processing device 100 then ends the processing shown in FIG. 9.

The medical information processing device 100 of the second embodiment achieves the same operations and effects as the medical information processing device 100 of the first embodiment. The medical information processing device 100 of the second embodiment further requests that an external device provide missing data that is lacking when generating a new workflow. This increases the number of types of new workflows that can be generated.

In the above embodiments, an example in which an existing workflow is updated to a new workflow has been described, but the medical information processing device 100 may also create a new workflow and compare the same with the existing workflow before registering the new workflow in the workflow DB 151. In this case, the medical information processing device 100 can display the update confirmation screen shown in FIG. 8 to provide the user with comparison information between the existing workflow and the new workflow. Therefore, in a case where a new application is released or the version of an existing application is upgraded, by generating a new workflow and displaying a result of comparison between the new workflow and the existing workflow, it is possible to provide the user with information to consider whether to introduce the new or upgraded application. The medical information processing device 100 may be provided within the medical image diagnostic device (modality) 30.

According to at least one of the above-described embodiments, the medical information processing device includes an acquisition unit that acquires update trigger information related to update of a workflow for processing medical information, and an update unit that updates the workflow on the basis of the update trigger information, thereby improving the efficiency of analysis work.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image diagnostic device that captures a medical image of a subject, comprising processing circuitry configured to:
acquire update trigger information related to an update of a workflow for processing the medical image; and
update the workflow on the basis of the update trigger information.

2. A medical information processing device comprising processing circuitry configured to:
acquire update trigger information related to an update of a workflow for processing medical information; and
update the workflow on the basis of the update trigger information.

3. The medical information processing device according to claim 2, wherein the update trigger information includes change information related to at least one change of an identification label included in the workflow, an application used, modality information related to a modality that has acquired the medical information, and workflow processing time, resources, accuracy, cost, number of executions, trend, or user reviews.

4. The medical information processing device according to claim 2, wherein the processing circuitry acquires the update trigger information every time a certain period or a designated period elapses.

5. The medical information processing device according to claim 2, wherein the processing circuitry provides comparison information indicating comparison of statuses before and after the workflow update, and updates the workflow on the basis of a response to the comparison information.

6. The medical information processing device according to claim 5, wherein the comparison information includes information indicating comparison of at least some of elements of the workflow before and after the update.

7. The medical information processing device according to claim 2, wherein the processing circuitry further analyzes the medical information in accordance with the workflow.

8. The medical information processing device according to claim 2, wherein the processing circuitry creates a new workflow to which missing data has been added, provides supplement request information for requesting supplement of the missing data, and acquires supplement information corresponding to the supplement request information.

9. A medical information processing system comprising:
the medical information processing device according to claim 8; and
an external device capable of communicating with the medical information processing device,
wherein the processing circuitry provides the supplement request information to the external device, and the external device provides the supplement request information to the medical information processing device.

10. The medical information processing system according to claim 9, wherein the supplement request information is medical images, and the external device includes an image storage device configured to store the plurality of medical images.

11. The medical information processing system according to claim 9, wherein the supplement request information includes examination information related to an examination, and the external device includes an examination device configured to perform the examination.

12. A medical information processing system comprising:
the medical information processing device according to claim 2; and
an external device capable of communicating with the medical information processing device and configured to manage an external workflow in which a processing procedure changes depending on the workflow,
wherein the processing circuitry provides information on an updated workflow to the external device.

13. A medical information processing method, using a computer, comprising:
acquiring update trigger information related to an update of a workflow for processing medical information; and
updating the workflow on the basis of the update trigger information.

14. A computer-readable non-transitory storage medium storing a program causing a computer to:
acquire update trigger information related to an update of a workflow for processing medical information; and
update the workflow on the basis of the update trigger information.
